(19) United States
(12) Patent Application Publication
(10) Pub. No.: US 2025/0271276 A1
(43) Pub. Date: Aug. 28, 2025

(54) NAVIGATION SYSTEM

(71) Applicant: Murata Manufacturing Co., Ltd., Kyoto (JP)

(72) Inventors: Toru TANAKA, Kyoto (JP); Hiroshi KUNIMATSU, Kyoto (JP); Yuuji NIHEI, Kyoto (JP)

(21) Appl. No.: 19/195,781

(22) Filed: May 1, 2025

Related U.S. Application Data
(63) Continuation of application No. PCT/JP2023/039283, filed on Oct. 31, 2023.

(30) Foreign Application Priority Data
Nov. 7, 2022 (JP) 2022-177823

(51) Int. Cl. G01C 21/36; G01C 21/00; G06F 3/01

(52) U.S. Cl. CPC G01C 21/3617; G01C 21/3694; G01C 21/3896; G06F 3/015

(57) ABSTRACT

Assist driving in a manner that depends on cognitive abilities. A navigation system includes a cognitive ability assessment part, a map information database, and a route search part. The cognitive ability assessment part assesses a cognitive ability of a driver relating to driving. The map information database stores map information for use in a route search. The route search part conducts the route search from a location of a vehicle to a destination location using a starting location of the route search, the destination location of the route search, and the map information.

### CROSS REFERENCE TO RELATED APPLICATION

This is a continuation of International Application No. PCT/JP2023/039283 filed on Oct. 31, 2023 which claims priority from Japanese Patent Application No. 2022-177823 filed on Nov. 7, 2022. The contents of these applications are incorporated herein by reference in their entireties.

### BACKGROUND OF THE DISCLOSURE

### Field of the Disclosure

The present disclosure relates to navigation systems that support driving in a manner that depends on cognitive abilities of drivers.

### Description of the Related Art

Japanese Unexamined Patent Application Publication No. 2008-058193 describes a navigation device. The navigation device in Japanese Unexamined Patent Application Publication No. 2008-058193 monitors driver's actions and accumulates monitoring results of the driver's actions.

By utilizing the accumulated monitoring results of the driver's actions, the navigation device in Japanese Unexamined Patent Application Publication No. 2008-058193 provides a route guidance service that aligns with inclinations of the driver's actions or characteristics of the driver.

Japanese Unexamined Patent Application Publication No. 2015-080549 describes a cognition degree estimation method. The recognition degree estimation method in Japanese Unexamined Patent Application Publication No. 2015-080549 detects a decline in the recognition degree while driving. The recognition degree estimation method in Japanese Unexamined Patent Application Publication No. 2015-080549 uses the detected decline in the recognition degree while driving to assist the driving.

### BRIEF SUMMARY OF THE DISCLOSURE

Currently, traffic accidents involving senior drivers and the like are becoming problematic. One factor contributing to this is driving by drivers with declined cognitive abilities.

However, the navigation device in Japanese Unexamined Patent Application Publication No. 2008-058193 and the cognitive degree estimation method in Japanese Unexamined Patent Application Publication No. 2015-080549 do not consider the cognitive abilities when assisting driving.

Accordingly, a possible benefit of the present disclosure is to assist driving in a manner that depends on a cognitive ability.

A navigation system according to an embodiment of this disclosure includes a cognitive ability assessment part, a map information database, and a route search part. The cognitive ability assessment part assesses a cognitive ability of a driver relating to driving. The map information database stores map information for use in a route search. The route search part conducts the route search from a location of a vehicle to a destination location using a starting location of the route search, the destination location of the route search, and the map information.

The map information database includes, as the map information, first map information that contains at least a plurality of roads for use in the route search as a plurality of constituent elements and second map information that contains weighting information grouped by the cognitive ability, the weighting information grouped by the cognitive ability being set for the plurality of constituent elements of the first map information on the basis of the cognitive ability.

The route search part conducts the route search on the basis of the first map information and the second map information.

With this configuration, the route is searched in a manner that depends on the cognitive ability.

According to the present disclosure, it becomes possible to assist driving in a manner that depends on the cognitive ability.

### BRIEF DESCRIPTION OF THE SEVERAL VIEWS OF THE DRAWINGS

FIG. 1 is a functional block diagram of a navigation system according to a first embodiment of the present disclosure.

FIG. 2A is a table illustrating one example of setting of cognitive ability levels.

FIG. 2B is a table illustrating one example of setting of weighting coefficients grouped by the cognitive ability.

FIG. 2C is a table illustrating an exemplary setting of the cognitive ability levels and the weighting coefficients for each road.

FIG. 3 is a functional block diagram of a cognitive ability assessment part according to the first embodiment of the present disclosure.

FIG. 4 is a functional block diagram of a navigation device according to the first embodiment of the present disclosure.

FIG. 5 is a flowchart illustrating a first example of a navigation method performed by the navigation system according to the first embodiment of the present disclosure.

FIG. 6 is a flowchart illustrating a second example of the navigation method performed by the navigation system according to the first embodiment of the present disclosure.

FIG. 7 is a functional block diagram of a navigation device of a navigation system according to a second embodiment of the present disclosure.

FIG. 8 is a flowchart illustrating a first example of an information feedback method performed by the navigation system according to the second embodiment of the present disclosure.

FIG. 9 is a flowchart illustrating a second example of the information feedback method performed by the navigation system according to the second embodiment of the present disclosure.

FIG. 10 is a flowchart illustrating a third example of the information feedback method performed by the navigation system according to the second embodiment of the present disclosure.

### DETAILED DESCRIPTION OF THE DISCLOSURE

### First Embodiment

A navigation system according to the first embodiment of the present disclosure is now described with reference to the drawings.

### System Configuration

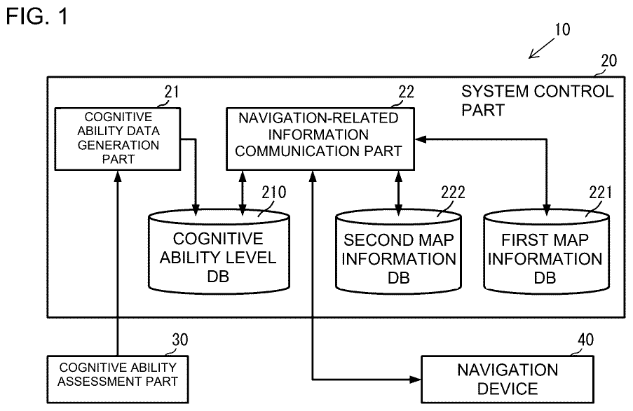

FIG. 1 is a functional block diagram of a navigation system according to the first embodiment of the present disclosure. As illustrated in FIG. 1, a navigation system 10 includes a system control part 20 (e.g., implemented as one or more processors), a cognitive ability assessment part 30, and a navigation device 40.

The system control part 20, the cognitive ability assessment part 30, and the navigation device 40 are connected to each other via an information communication network such as the Internet or the like.

The system control part 20 is included in a device that can be connected to the information communication network or on the cloud (e.g., remote form a vehicle) such as a cloud server or the like.

The cognitive ability assessment part 30 is incorporated in a device worn by a driver. Here, in most cases, the driver means a person who is ready to drive a vehicle but is not currently driving the vehicle. However, the driver may also mean a person who is currently driving a vehicle.

The navigation device 40 is attached to the vehicle that the driver drives.

The navigation system 10 generally performs the following processes.

The cognitive ability assessment part 30 measures, before the route search, brain waves of a driver and generates cognitive ability assessment data. The cognitive ability assessment data is data for assessment of a cognitive ability (particularly, a potential cognitive ability) of the driver relating to driving. The cognitive ability assessment part 30 outputs the cognitive ability assessment data to the system control part 20.

The system control part 20 stores in advance map information for use in general route searches (first map information) and map information containing weighting information grouped by the cognitive ability that is associated with the cognitive ability level (second map information).

The system control part 20 assesses a cognitive ability of a driver relating to driving and generates a cognitive ability level by utilizing the cognitive ability assessment data.

The system control part 20 receives a starting location, a destination location, and identification information of a driver from the navigation device 40. On the basis of a variety of information from the navigation device 40, the system control part 20 detects the cognitive ability level of the driver from the identification information of the driver. The system control part 20 supplies the first map information and the second map information associated with the detected cognitive ability level to the navigation device 40.

The navigation device 40 performs the route search in a manner that depends on the cognitive ability by utilizing the information supplied from the system control part 20.

Because of this, the navigation system 10 can assist driving in a manner that depends on the cognitive ability of the driver.

### System Control Part 20

As illustrated in FIG. 1, the system control part 20 includes a cognitive ability data generation part 21, a navigation-related information communication part 22, a cognitive ability level database (cognitive ability level DB) 210, a first map information database (first map information DB) 221, and a second map information database (second map information DB) 222. The first map information database 221 and the second map information database 222 are included separately. In other words, the navigation-related information communication part 22 can independently access both the first map information database 221 and the second map information database 222.

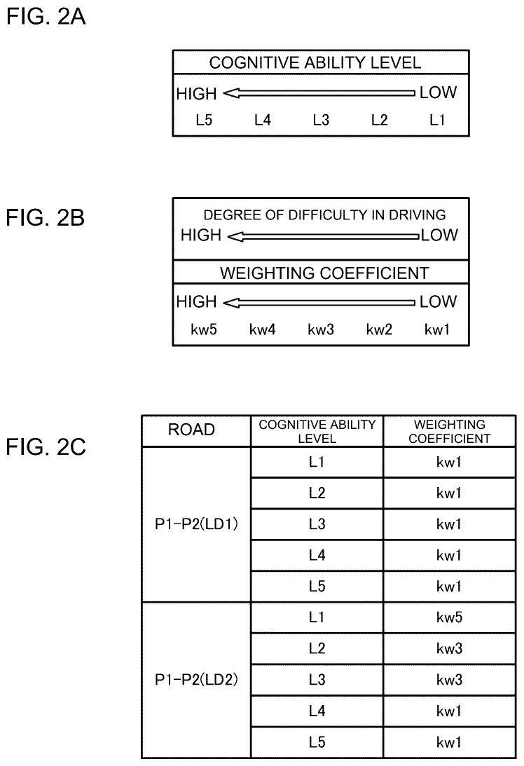

FIG. 2A is a table illustrating one example of setting of cognitive ability levels, FIG. 2B is a table illustrating one example of setting of weighting coefficients grouped by the cognitive ability, and FIG. 2C is a table illustrating an exemplary setting of the cognitive ability levels and the weighting coefficients for each road.

The cognitive ability data generation part 21 acquires data for cognitive ability assessment from the cognitive ability assessment part 30. The cognitive ability data generation part 21 estimates the cognitive ability level of the driver by utilizing the data for cognitive ability assessment. The cognitive ability data generation part 21 estimates the cognitive ability level out of a plurality of discrete levels. For example, the cognitive ability data generation part 21 estimates the cognitive ability level on a scale of five levels and sets the cognitive ability level out of a plurality of cognitive ability levels L1, L2, L3, L4, and L5. Here, as one example, as illustrated in FIG. 2A, the plurality of cognitive ability levels L1, L2, L3, L4, and L5 are set in such a way that the cognitive ability increases as the index of “L” increases. In other words, the plurality of cognitive ability levels L1, L2, L3, L4, and L5 are set in such a way that the decline in cognitive ability progresses as the index of “L” decreases.

The cognitive ability data generation part 21 generates cognitive ability data by linking the cognitive ability level to the driver (driver ID) who is the subject of this cognitive ability level estimation. The cognitive ability data generation part 21 stores the cognitive ability data in the cognitive ability level database 210.

The estimation of the cognitive ability level of the driver and the generation of the cognitive ability data, such as the ones described above, are conducted at predetermined intervals (for example, every six months, annually, or the like). Every time new cognitive ability data is generated, the cognitive ability data generation part 21 updates the cognitive ability data and stores the latest cognitive ability data in the cognitive ability level database 210.

The cognitive ability level database 210 stores therein the cognitive ability data for each driver.

The first map information database 221 stores therein the first map information for use in general route searches. The first map information contains, as first constituent elements, a plurality of roads and connection points of the plurality of roads. Furthermore, the first map information contains, as the first constituent elements, for example, distances of the plurality of roads, types (ordinary road or toll road) of the plurality of roads, information regarding intersections where the plurality of roads cross (priority road and the like), widths of the plurality of roads, traffic congestion information, and traffic information (traffic congestion information, speed limits, and the like). The first constituent elements correspond to “external caution information”.

Note that the first map information only needs to contain at least a plurality of roads and connection points of the plurality of roads and only needs to contain other constituent elements as required depending on necessary specifications for route searches or the like.

Further, the first map information sets an index value for the route search for each of the first constituent elements such as the roads and the like. For example, in the case of the distance of a road, the index value for the route search is set in such a way that the index value for the route search increases as the distance of the road decreases.

The second map information database 222 stores therein the second map information that reflects the cognitive ability. The second map information contains the weighting information grouped by the cognitive ability that is respectively set for the plurality of roads and the connection points of the plurality of roads on the basis of the cognitive ability level.

The weighting information grouped by the cognitive ability is information that sets the priority in the road selection in the route search in a manner that depends on the cognitive ability. For example, as illustrated in FIG. 2B, the second map information database 222 sets the weighting coefficient out of weighting coefficients kw1, kw2, kw3, kw4, and kw5 in a manner that depends on the degree of difficulty in driving on each of the plurality of roads. The weighting coefficients kw1, kw2, kw3, kw4, and kw5 are set in such a way that as the degree of difficulty in driving for a road increases, the weighting coefficient increases and the index of “kw” increases (kw1<kw2<kw3<kw4<kw5). The weighting coefficient is set, for example, as a positive value.

For example, the degree of difficulty in driving is set on the basis of the following items in such a way that the degree of difficulty in driving increases as the number of the following items contained increases.
Road including a location where many traffic accidents occurred.
Road included in a route with many left turns or right turns
Road including many stop signs
Road including a dark tunnel
Road with many connecting side streets
Road including many lanes and many entryways
Road in which the distance between intersections or the installation distance between traffic lights is short
Road with heavy traffic of large vehicles
Road with extensive on-street parking

Further, for example, as illustrated in FIG. 2C, the second map information database 222 sets a weighting coefficient for each of the plurality of roads for each of the cognitive ability levels L1, L2, L3, L4, and L5. The weighting coefficients set for the corresponding roads in a manner that depends on the cognitive ability level are the weighting coefficients grouped by the cognitive ability.

For example, in the case of FIG. 2C, in a road LD1 that connects a point P1 and a point P2, the degree of difficulty in driving is the lowest for drivers of all the cognitive ability levels (L1, L2, L3, L4, and L5). Accordingly, the weighting coefficient kw1 is set for all the cognitive ability levels (L1, L2, L3, L4, and L5).

On the other hand, in a road LD2 that connects the point P1 and the point P2, the degree of difficulty in driving is the highest for the driver of the cognitive ability level L1. Accordingly, the weighting coefficient kw5 is set for the cognitive ability level L1. Further, in the road LD2, the degree of difficulty in driving is moderately high for the drivers of the cognitive ability levels L2 and L3. Accordingly, the weighting coefficient kw3 is set for the cognitive ability levels L2 and L3. Further, in the road LD2, the degree of difficulty in driving is low for the drivers of the cognitive ability levels L4 and L5. Accordingly, the weighting coefficient kw1 is set for the cognitive ability levels L4 and L5.

Note that the setting of the weighting coefficients in a manner that depends on the cognitive ability level is not limited to the method described above. The weighting coefficients only need to be set in such a way that a road with a high degree of difficulty in driving would not be selected for a person with a low cognitive ability level.

The navigation-related information communication part 22 receives the starting location, the destination location, and the identification information of the driver from the navigation device 40. The navigation-related information communication part 22 detects the cognitive ability level of the driver by referencing the cognitive ability level database 210 on the basis of the identification information of the driver. The navigation-related information communication part 22 supplies first map information based on the starting location and the destination location and second map information based on the first map information and the cognitive ability level to the navigation device 40.

In this way, the navigation-related information communication part 22 can supply the map information for the route search to the navigation device 40 in such a manner that depends on the cognitive ability of the driver relating to driving.

### Cognitive Ability Assessment Part 30

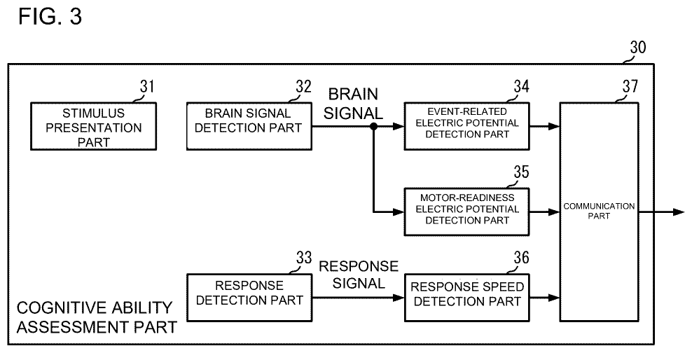

FIG. 3 is a functional block diagram of the cognitive ability assessment part according to the first embodiment of the present disclosure. The cognitive ability assessment part 30 includes a stimulus presentation part 31, a brain signal detection part 32, a response detection part 33, an event-related electric potential detection part 34, a motor-readiness electric potential detection part 35, a response speed detection part 36, and a communication part 37.

The cognitive ability assessment part 30 generates cognitive ability assessment data in a way described below before the driver starts driving, in other words, before conducting the route search.

The stimulus presentation part 31 presents a stimulus to the driver who is the subject of the cognitive ability assessment. The stimulus is, for example, a stimulus simulating an image or sound related to a hazard while driving.

The brain signal detection part 32 is, for example, a wearable terminal and worn by the driver who is the subject of the cognitive ability assessment. The brain signal detection part 32 detects a brain signal of this driver. The brain signal detection part 32 outputs the brain signal to the event-related electric potential detection part 34 and the motor-readiness electric potential detection part 35.

The event-related electric potential detection part 34 detects an event-related electric potential (P300 or the like) from the brain signal. In the case of a visual stimulus, the event-related electric potential is a visual event-related electric potential (for example, P300), and in the case of an auditory stimulus, the event-related electric potential is an auditory event-related electric potential (for example, MMN). The event-related electric potential detection part 34 outputs a detection result of the event-related electric potential (presence or absence of the event-related electric potential and the like) to the communication part 37.

The motor-readiness electric potential detection part 35 detects a motor-readiness electric potential from the brain signal. The motor-readiness electric potential detection part 35 outputs a detection result of the motor-readiness electric potential (presence or absence of the motor-readiness electric potential, time from the stimulus presentation to the occurrence of the motor-readiness electric potential, and the like) to the communication part 37.

The response detection part 33 is a simulated steering wheel, a simulated pedal, an operation button, or the like, which is a device that detects a response operation of the driver, who is the subject of the cognitive ability assessment, to a stimulus. The response detection part 33 detects the response operation of the driver who is the subject of the cognitive ability assessment and generates a response signal. The response detection part 33 outputs the response signal to the response speed detection part 36.

The response speed detection part 36 detects a response speed from the time difference between timing of the stimulus presentation and detection timing of the response signal. The response speed detection part 36 outputs the response speed to the communication part 37.

The communication part 37 generates cognitive ability assessment data that includes at least one of the detection result of the event-related electric potential, the detection result of the motor-readiness electric potential, and the response speed. The communication part 37 links the cognitive ability assessment data to the driver ID (identification information of the driver) having been assessed and sends the cognitive ability assessment data and the driver ID to the cognitive ability data generation part 21 of the system control part 20.

Note that in the example described above, the case is described using the visual stimulus. Alternatively, an auditory stimulus may be used, or both the visual stimulus and the auditory stimulus may be used.

As described above, the cognitive ability assessment data is based on the event-related electric potential or the motor-readiness electric potential that uses the brain signal (brain waves) formed in response to the visual stimulus or the auditory stimulus. Accordingly, the navigation system 10 can assess the cognitive ability of the driver relating to driving with a high degree of accuracy. Furthermore, the cognitive ability assessment data is based on the response speed. Accordingly, the navigation system 10 can assess the cognitive ability of the driver relating to driving with a high degree of accuracy.

### Navigation Device 40

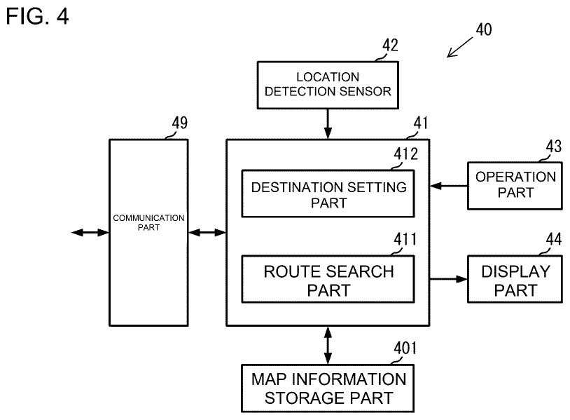

FIG. 4 is a functional block diagram of the navigation device according to the first embodiment of the present disclosure. As illustrated in FIG. 4, the navigation device 40 includes a navigation control part 41, a location detection sensor 42, an operation part 43, a display part 44, a communication part 49, and a map information storage part 401. The navigation control part 41 includes a route search part 411 and a destination setting part 412.

The communication part 49 conducts information communication with an external device located outside the navigation device 40 including the system control part 20.

The map information storage part 401 stores therein map information for displaying the current location of a vehicle, a starting location, a destination location, and a route search result on the display part 44. Between the map information of the map information storage part 401 and the first map information, identification information of roads and the like are linked.

The location detection sensor 42 is realized by, for example, a GPS location sensor and detects the current location of the vehicle. The location detection sensor 42 outputs the detected current location to the navigation control part 41.

The operation part 43 receives an operation input for the route search. For example, the operation part 43 is realized by a touch panel incorporated in the display part 44.

The display part 44 displays a map stored in the map information storage part 401. Further, the display part 44 displays the current location of the vehicle. Furthermore, the display part 44 displays navigation information. The navigation information contains a recommended route acquired from a result of the route search conducted by the navigation control part 41.

The destination setting part 412 of the navigation control part 41 sets a destination location of the route search, for example, on the basis of an input result of the operation of the operation part 43.

The route search part 411 of the navigation control part 41 acquires the current location of the vehicle from the location detection sensor 42 and acquires the destination location of the route search from the destination setting part 412. The route search part 411 acquires the starting location when the starting location is specified using the operation part 43, for example.

Further, the route search part 411 acquires the identification information of the driver who is currently driving the vehicle. The identification information of the driver can be acquired through various methods. For example, the identification information of the driver can be acquired using the input operation of the operation part 43, a contact-based target identification sensor for detecting a target subject, or the like.

The route search part 411 sends the starting location of the route search (or the current location of the vehicle), the destination location of the route search, and the identification information of the driver to the navigation-related information communication part 22 of the system control part 20 via the communication part 49.

As described above, the navigation-related information communication part 22 supplies the first map information and the second map information for use in the route search to the route search part 411 on the basis of the starting location of the route search (or the current location of the vehicle), the destination location of the route search, and the identification information of the driver.

The route search part 411 conducts the route search using the first map information and the second map information. For example, the route search part 411 conducts the route search using the Dijkstra's algorithm or an equivalent search method. In this case, the route search part 411 acquires index values for the first constituent elements such as roads (edges), intersections (nodes), and the like from the first map information. The route search part 411 acquires the weighting coefficients (weighting information grouped by the cognitive ability) associated with the cognitive ability level set for the roads (edges) and the intersections (nodes).

For example, the route search part 411 multiplies the index value by the weighting coefficient and conducts the route search using a result of the multiplication.

Performing the processes described above enables the navigation system 10 to conduct the route search in a manner that depends on the cognitive ability of the driver. Specifically, for example, the navigation system 10 enables a person with low cognitive ability to select a route with a lower degree of difficulty in driving, even when there is an available route that would normally be prioritized in standard route searches. On the other hand, the navigation system 10 enables a person with high cognitive ability to select a route that would normally be prioritized in the standard route searches.

Note that in the configuration described above, the example illustrates that the functional part conducting the route search (route search part 411) is included in the navigation device 40. Alternatively, the functional part conducting the route search (route search part) may be included in the system control part 20.

In this case, the navigation device 40 sends the current location of the vehicle and the destination location of the route search to the system control part 20. As is the case with the route search part 411 described above, a route search part of the system control part 20 conducts the route search using the current location of the vehicle and the destination location of the route search sent from the navigation device 40. Further, the system control part 20 sends a result of the route search to the navigation device 40.

### Navigation Method 1

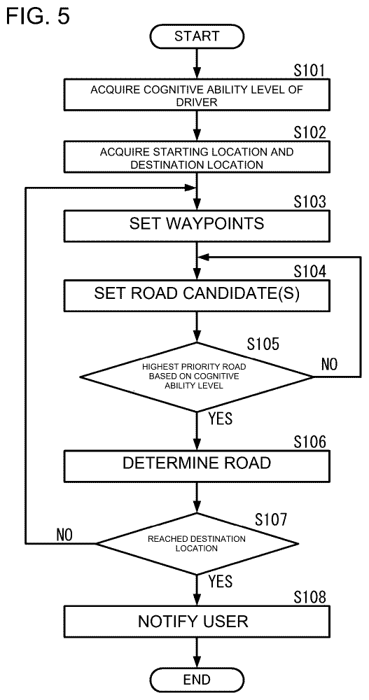

FIG. 5 is a flowchart illustrating a first example of a navigation method performed by the navigation system according to the first embodiment of the present disclosure. Note that the details of the respective processes are described above, and the detailed descriptions thereof are omitted below.

The navigation system 10 acquires the cognitive ability level of the driver (S101). In this case, the navigation system 10 acquires the cognitive ability level before the driver starts driving, in other words, before conducting the route search.

The navigation system 10 acquires the starting location of the route search and the destination location of the route search (S102).

The navigation system 10 sets a plurality of waypoints in between the starting location and the destination location (S103).

The navigation system 10 conducts the route search using the Dijkstra's algorithm or an equivalent search method. Specifically, the navigation system 10 conducts the following individual processes, which are described conceptually.

The navigation system 10 sets one or more road candidates (S104). When there is a plurality of road candidates, the navigation system 10 selects one road candidate and conducts an adjustment in a manner that depends on the cognitive ability level (adjustment using the weighting coefficients grouped by the cognitive ability). When the selected road candidate that has undergone the adjustment is the highest priority road (S105: YES), the navigation system 10 determines this road candidate as the road to be selected (S106). Note that when the selected road candidate that has undergone the adjustment is not the highest priority road (S105: NO), the navigation system 10 selects a different road and conducts the same process.

When the destination location has not been reached (S107: NO), the navigation system 10 sets the next waypoint and continues the process of determining the road to be selected.

When the destination location is reached (S107: YES), the navigation system 10 notifies a user (for example, the driver) of a recommended route from the starting location to the destination (S108). As the notification, the navigation system 10 conducts a process of adding the recommended route on a map displayed on the display part 44, for example.

Note that in the method described above, the case is described where the cognitive ability assessment is conducted before the route search. However, a route search can be conducted on the basis of the cognitive ability assessed after a route search.

For example, the cognitive ability assessment part 30 assesses the cognitive ability after a route search. The map information database updates the second map information on the basis of the cognitive ability assessed after the route search. The route search part 411 conducts a route search on the basis of the updated second map information.

This enables the navigation system 10 to conduct the route search in a manner that depends on the latest cognitive ability.

### Navigation Method 2

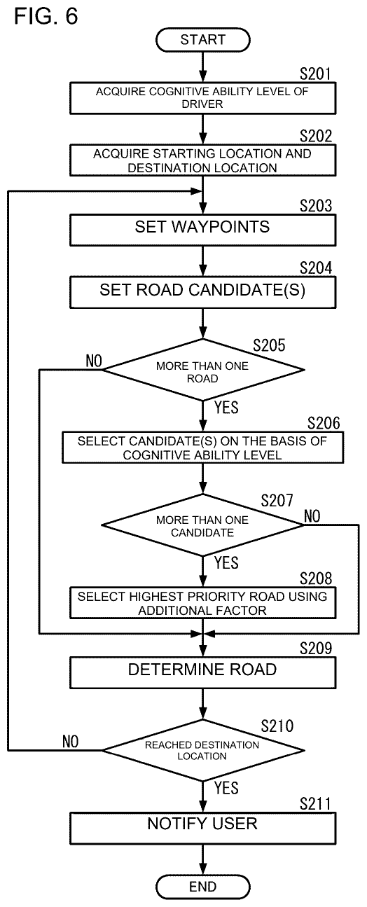

FIG. 6 is a flowchart illustrating a second example of the navigation method performed by the navigation system according to the first embodiment of the present disclosure.

The second example is another example in which a plurality of highest priority roads exists when the adjustment based on the cognitive ability level is conducted in the first example.

The navigation system 10 acquires the cognitive ability level of the driver (S201). In this case, the navigation system 10 acquires the cognitive ability level before the driver starts driving, in other words, before conducting the route search.

The navigation system 10 acquires the starting location of the route search and the destination location of the route search (S202).

The navigation system 10 sets a plurality of waypoints in between the starting location and the destination location (S203).

The navigation system 10 sets one or more road candidates (S204). When there is a plurality of road candidates (S205: YES), the navigation system 10 selects one road candidate and conducts the adjustment in a manner that depends on the cognitive ability level (adjustment using the weighting coefficients grouped by the cognitive ability). When the selected road candidate that has undergone the adjustment is the highest priority road, in other words, when only one highest priority road exists (S207: NO), the navigation system 10 determines this road candidate as the road to be selected (S209).

When a plurality of highest priority roads exists (S207: YES), the navigation system 10 selects one of the plurality of highest priority roads using an additional factor (S208). The additional factor is, for example, the road surface condition of each road caused by weather or the like, traffic congestion information, or the like.

When the destination location has not been reached (S210: NO), the navigation system 10 sets the next waypoint and continues the process of determining the road to be selected.

When the destination location is reached (S210: YES), the navigation system 10 notifies a user (for example, the driver) of a recommended route from the starting location to the destination (S211).

Note that the additional factor may be contained in advance in the first map information. In this case, the navigation system 10 may select any one of a plurality of highest priority roads as appropriate or may select one of the plurality of highest priority roads on the basis of an assessment result of the road next to the plurality of highest priority roads.

### Second Embodiment

A navigation system according to the second embodiment of the present disclosure is now described with reference to the drawings. The navigation system according to the second embodiment is different from the navigation system according to the first embodiment in the configuration of the navigation device and the processes of the system control part. The remaining configuration and processes of the navigation system according to the second embodiment are similar to those of the navigation system 10 according to the first embodiment, and the descriptions regarding the similar parts are omitted.

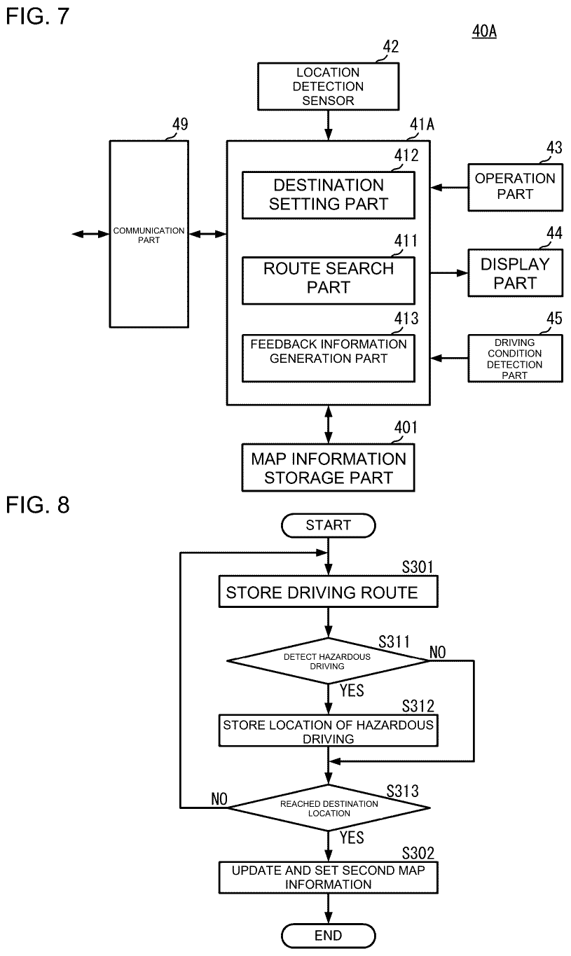

FIG. 7 is a functional block diagram of the navigation device of the navigation system according to the second embodiment of the present disclosure. As illustrated in FIG. 7, a navigation device 40A is different from the navigation device 40 in including a navigation control part 41A and a driving condition detection part 45. The navigation control part 41A is different from the navigation control part 41 according to the first embodiment in including a feedback information generation part 413.

The driving condition detection part 45 detects the driving condition of the driver. The driving condition contains at least one of hazardous driving and route deviation. The hazardous driving contains at least one of sudden starting, sudden steering, sudden stopping, wrong-way driving, and stop sign violation of the vehicle. The route deviation means that the actual route of the vehicle based on the current location of the vehicle is different from the recommended route (recommended route) presented to the driver by the route search.

The driving condition detection part 45 outputs the detected driving condition to the feedback information generation part 413 of the navigation control part 41A.

In the case of the hazardous driving, the feedback information generation part 413 generates feedback information containing the type of the hazardous driving, the location of the hazardous driving, and the cognitive ability level of the driver. In the case of the route deviation, the feedback information generation part 413 generates feedback information containing the location of the route deviation, the condition of the route deviation (to which direction the vehicle deviates), and the cognitive ability level of the driver.

The feedback information generation part 413 sends the feedback information to the navigation-related information communication part 22 of the system control part 20.

The navigation-related information communication part 22 analyzes the feedback information and updates the second map information. Specifically, in the case of the hazardous driving, the navigation-related information communication part 22 extracts the type and location of the hazardous driving from the feedback information and then updates and sets the degree of difficulty in driving on the road that corresponds to the location. Further, the navigation-related information communication part 22 updates and sets the weighting coefficients associated with the cognitive ability level (weighting information grouped by the cognitive ability) in the second map information using the cognitive ability level extracted from the feedback information and the cognitive ability level already stored in the second map information.

Because of the configurations and processes described above, the navigation system can adjust the second map information more accurately in relation to the cognitive ability level. Because of this, in the second map information, the weighting coefficients more accurately reflecting the cognitive ability level are set.

Accordingly, the navigation system can assist the driving more appropriately in consideration of the cognitive ability.

### Information Feedback Method 1

FIG. 8 is a flowchart illustrating a first example of an information feedback method performed by the navigation system according to the second embodiment of the present disclosure. FIG. 8 illustrates the first example of the information feedback method based on the hazardous driving.

The navigation system detects hazardous driving (S311) while storing the driving route (S301). When the navigation system detects the hazardous driving (S311: YES), the navigation system stores the location of the hazardous driving (S312).

The navigation system repeats the processes described above until the destination location is reached (S313: NO).

When the destination location is reached (S313: YES), the navigation system generates the feedback information using a detection result of the hazardous driving and the location of the hazardous driving. The navigation system updates and sets the second map information on the basis of the feedback information (S302).

### Information Feedback Method 2

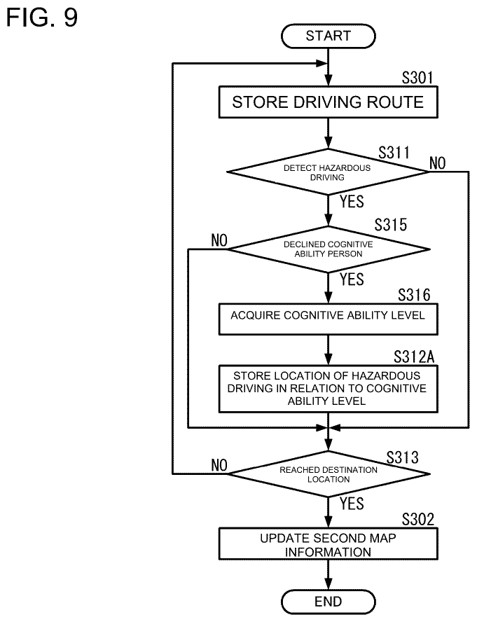

FIG. 9 is a flowchart illustrating a second example of the information feedback method performed by the navigation system according to the second embodiment of the present disclosure. FIG. 9 illustrates the second example of the information feedback method based on the hazardous driving. The second example is different from the first example in that the cognitive ability level of the driver is checked every time the hazardous driving is detected.

The navigation system detects hazardous driving (S311) while storing the driving route (S301). When the driving condition detection part 45 detects the hazardous driving (S311: YES), the navigation control part 41A (that corresponds to “cognitive-ability-while-driving detection part”) of the navigation device 40A detects the cognitive ability level of the driver. For example, the navigation control part 41A acquires the cognitive ability level of the driver from the cognitive ability level DB 210 of the system control part 20. When the navigation system detects that the driver is a declined cognitive ability person (person with a declined cognitive ability level) (S315: YES), the navigation system acquires the stored cognitive ability level of the driver (S316). Note that in this case, if it is possible to conduct a brain wave measurement for assessing the cognitive ability level of the driver, the navigation control part 41A may detect the cognitive ability level while driving and use this detected cognitive ability level.

When the driver is a declined cognitive ability person, the navigation system stores the detection result of the hazardous driving, the location of the hazardous driving, and the cognitive ability level by linking them together (S312A).

When the navigation system detects that the driver is not a declined cognitive ability person (S315: NO), the navigation system does not store the location of the hazardous driving.

The navigation system repeats the respective processes described above until the destination location is reached (S313: NO).

When the destination location is reached (S313: YES), the navigation system generates the feedback information using a detection result of the hazardous driving, the location of the hazardous driving, and the cognitive ability level. The navigation system updates and sets the second map information on the basis of the feedback information (S302).

### Information Feedback Method 3

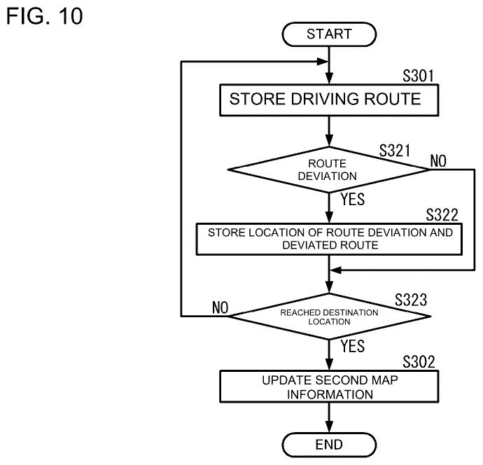

FIG. 10 is a flowchart illustrating a third example of the information feedback method performed by the navigation system according to the second embodiment of the present disclosure. FIG. 10 illustrates one example of the information feedback method based on the route deviation.

The navigation system detects route deviation (S321) while storing the driving route (S301). When the navigation system detects the route deviation (S321: YES), the navigation system stores the location of the route deviation and a deviated route (road mistakenly drove) (S322).

The navigation system repeats the processes described above until the destination location is reached (S323: NO).

When the destination location is reached (S323: YES), the navigation system generates the feedback information using a detection result of the route deviation, the location of the route deviation, and the deviated route. The navigation system updates and sets the second map information on the basis of the feedback information (S302).

Note that in each of the embodiments described above, the navigation system may send an alarm notification when approaching a road whose degree of difficulty in driving is high or an intersection. In this case, the navigation system may select whether or not to send the alarm notification in a manner that depends on the cognitive ability level. The navigation system may make a selection regarding the alarm notification. For example, the navigation system may send an alarm notification only when the cognitive ability level is equal to or lower than a predetermined level and send no alarm notification when the cognitive ability level exceeds the predetermined level.

<1> A navigation system comprising: a cognitive ability assessment part that assesses a cognitive ability of a driver relating to driving; a map information database that stores map information for use in a route search; and a route search part that conducts the route search from a starting location to a destination location using the starting location of the route search, the destination location of the route search, and the map information, wherein the map information database includes, as the map information, first map information that contains at least a plurality of roads for use in the route search as a plurality of constituent elements, and second map information that contains weighting information grouped by the cognitive ability, the weighting information grouped by the cognitive ability being set for the plurality of constituent elements of the first map information on a basis of the cognitive ability, and the route search part conducts the route search on a basis of the first map information and the second map information.

<2> The navigation system of <1>, wherein the map information database contains external caution information at a time of driving in the first map information, and the route search part conducts the route search using the first map information containing the external caution information.

<3> The navigation system of <1> or <2>, wherein the map information database is included in a system control part on a cloud, the route search part is attached to a vehicle that the driver drives, and the route search part conducts the route search by acquiring the first map information and part of the second map information from the map information database.

<4> The navigation system of <1> or <2>, wherein the map information database and the route search part are included in a system control part on a cloud, and the route search part conducts the route search by acquiring a current location of a vehicle and a destination location of the route search from the vehicle that the driver drives.

<5> The navigation system of any one of <1> to <3>, wherein the map information database includes a first map information database that stores the first map information, and a second map information database that stores the second map information, and the first map information database and the second map information database are included separately.

<6> The navigation system of any one of <1> to <5>, further comprising: a driving condition detection part that detects a condition of the driving; and a feedback information generation part, wherein when the driving condition detection part detects hazardous driving of the vehicle as the condition of the driving, the driving condition detection part outputs a detection result to the feedback information generation part, the feedback information generation part generates feedback information that contains a location where the hazardous driving occurs and the cognitive ability of the driver and outputs the feedback information to the map information database, and the map information database updates the weighting information grouped by the cognitive ability using the cognitive ability of the driver contained in the feedback information and a cognitive ability level already stored in the second map information.

<7> The navigation system of <6>, wherein the feedback information contains at least one of sudden starting, sudden steering, sudden stopping, wrong-way driving, and stop sign violation of the vehicle.

<8> The navigation system of any one of <1> to <7>, further comprising: a feedback information generation part, wherein when a route presented to the driver as a result of the route search differs from an actual route of the vehicle based on a current location of the vehicle, the route search part outputs to the feedback information generation part, the feedback information generation part generates feedback information that contains a result of route deviation and the cognitive ability of the driver and outputs the feedback information to the map information database, and the map information database updates the weighting information grouped by the cognitive ability using the cognitive ability of the driver contained in the feedback information and a cognitive ability level already stored in the second map information.

<9> The navigation system of any one of <1> to <8>, further comprising: a cognitive-ability-while-driving detection part that detects a cognitive ability of the driver while driving, wherein the route search part conducts the route search further using the cognitive ability detected while driving.

<10> The navigation system of <6> or <7>, further comprising: a cognitive-ability-while-driving detection part that detects a cognitive ability of the driver while driving, wherein the route search part causes the hazardous driving information to further contain the cognitive ability detected while driving.

<11> The navigation system of any one of <1> to <10>, wherein the cognitive ability assessment part assesses the cognitive ability before the route search.

<12> The navigation system of any one of <1> to <11>, wherein the cognitive ability assessment part assesses the cognitive ability after the route search, the map information database updates the second map information on a basis of the cognitive ability assessed after the route search, and the route search part conducts the route search on a basis of the second map information updated.

<13> The navigation system of any one of <1> to <12>, wherein the cognitive ability assessment part includes a brain signal detection part that detects a brain signal formed in response to a visual stimulus or an auditory stimulus, and an event-related electric potential detection part that detects an event-related electric potential using the brain signal, and the cognitive ability assessment part assesses the cognitive ability using the event-related electric potential.

<14> The navigation system of <13>, wherein the cognitive ability assessment part further includes a motor-readiness electric potential detection part that detects a motor-readiness electric potential using the brain signal, and the cognitive ability assessment part assesses the cognitive ability using the event-related electric potential and the motor-readiness electric potential.

<15> The navigation system of <13> to <14>, wherein the cognitive ability assessment part further includes a response speed detection part that detects a response speed for the visual stimulus or the auditory stimulus, and the cognitive ability assessment part assesses the cognitive ability using the event-related electric potential, the motor-readiness electric potential, and the response speed.
10 Navigation system
20 System control part
21 Cognitive ability data generation part
22 Navigation-related information communication part
30 Cognitive ability assessment part
31 Stimulus presentation part
32 Brain signal detection part
33 Response detection part
34 Event-related electric potential detection part
35 Motor-readiness electric potential detection part
36 Response speed detection part
37 Communication part
40, 40A Navigation device
41, 41A Navigation control part
42 Location detection sensor
43 Operation part
44 Display part
45 Driving condition detection part
49 Communication part
210 Cognitive ability level database
221 First map information database
222 Second map information database
401 Map information storage part
411 Route search part
412 Destination setting part
413 Feedback information generation part

1. A navigation system for a vehicle comprising:
a map information database that stores map information for use in a route search; and
one or more processors collectively configured to: assess a cognitive ability of a driver relating to driving, and that conduct the route search from a starting location to a destination location using the starting location of the route search, the destination location of the route search, and the map information,
wherein the map information database includes, as the map information:
first map information that contains at least a plurality of roads for use in the route search as a plurality of constituent elements, and
second map information that contains weighting information grouped by the cognitive ability, the weighting information grouped by the cognitive ability being set for the plurality of constituent elements of the first map information on a basis of the cognitive ability, and
wherein the route search is conducted on a basis of the first map information and the second map information.

2. The navigation system according to claim 1,
wherein the map information database contains external caution information at a time of driving in the first map information, and
wherein the route search is conducted using the first map information containing the external caution information.

3. The navigation system according to claim 1,
wherein the map information database is remote from the vehicle,
wherein the route search part is attached to a vehicle that the driver drives, and
wherein the route search part conducts the route search by acquiring the first map information and part of the second map information from the map information database.

4. The navigation system according to claim 1,
wherein the map information database and the one or more processors are at least partially remote from the vehicle, and
wherein the route search is conducted by acquiring a current location of a vehicle and a destination location of the route search from the vehicle that the driver drives.

5. The navigation system according to claim 1,
wherein the map information database includes:
a first map information database that stores the first map information, and
a second map information database that stores the second map information, and
wherein the first map information database and the second map information database are separate.

6. The navigation system according to claim 1,
wherein the one or more processors are further collectively configured to:
detect a condition of the driving;
output a detection result when a hazardous driving of the vehicle is detected as the condition of the driving,
generate feedback information that contains a location where the hazardous driving occurs and the cognitive ability of the driver, and
output the feedback information to the map information database, and
wherein the map information database updates the weighting information grouped by the cognitive ability using the cognitive ability of the driver contained in the feedback information and a cognitive ability level already stored in the second map information.

7. The navigation system according to claim 6, wherein
the hazardous driving determination is based on a sudden starting, sudden steering, sudden stopping, wrong-way driving, or stop sign violation of the vehicle.

8. The navigation system according to claim 1,
wherein the one or more processors are further collectively configured to:
determine a route deviation when a route presented to the driver as a result of the route search differs from an actual route of the vehicle based on a current location of the vehicle,
generate feedback information that contains a result of route deviation and the cognitive ability of the driver, and
output the feedback information to the map information database, and
wherein the map information database updates the weighting information grouped by the cognitive ability using the cognitive ability of the driver contained in the feedback information and a cognitive ability level already stored in the second map information.

9. The navigation system according to claim 1, wherein the one or more processors are further collectively configured to:
detect a cognitive ability of the driver while driving, and
conduct the route search further using the cognitive ability detected while driving.

10. The navigation system according to claim 6, wherein the one or more processors are further collectively configured to detect a cognitive ability of the driver while driving.

11. The navigation system according to claim 1, wherein the cognitive ability is assessed before the route search.

12. The navigation system according to claim 1,
wherein the cognitive ability is assessed after the route search,
wherein the map information database updates the second map information on a basis of the cognitive ability assessed after the route search, and
wherein the route search is conducted on a basis of the second map information updated.

13. The navigation system according to claim 1,
wherein the one or more processors are further collectively configured to:
detect a brain signal formed in response to a visual stimulus or an auditory stimulus, and
detect an event-related electric potential using the brain signal, and
wherein the cognitive ability is assessed using the event-related electric potential.

14. The navigation system according to claim 13,
wherein the one or more processors are further collectively configured to detect a motor-readiness electric potential using the brain signal, and
wherein the cognitive ability is assessed using the event-related electric potential and the motor-readiness electric potential.

15. The navigation system according to claim 13,
wherein the one or more processors are further collectively configured to detect a response speed for the visual stimulus or the auditory stimulus, and
wherein the cognitive ability is assessed using the event-related electric potential, a motor-readiness electric potential, and the response speed.
